(19) United States
(12) Patent Application Publication
(10) Pub. No.: US 2023/0395191 A1
(43) Pub. Date: Dec. 7, 2023

(54) DRUG RESISTANCE OF TARGET STRAINS OF A PATHOGEN

(71) Applicant: AAROGYAAI INNOVATIONS PVT. LTD., Bangalore (IN)

(72) Inventors: Praapti JAYASWAL, New Delhi (IN); Avlokita TIWARI, New Delhi (IN)

(21) Appl. No.: 18/250,190

(22) PCT Filed: Nov. 30, 2021
(86) PCT No.: PCT/IN2021/051128

§ 371 Date: Apr. 21, 2023

(30) Foreign Application Priority Data
Dec. 21, 2020 (IN) 202041055641

(51) Int. Cl. G16B 20/20; G06N 20/00; G16B 30/10; G16B 40/20; G16H 20/10

(52) U.S. Cl. CPC G16B 20/20; G06N 20/00; G16B 30/10; G16B 40/20; G16H 20/10

(57) ABSTRACT

Examples of determining drug resistance of a target strain of a pathogen against a target drug are described. In an example, a nucleotide sequence data of the target strain of the pathogen is obtained from a test sample. The nucleotide sequence data may then be analyzed to locate a genetic variation in a nucleotide sequence of the target strain. Based on a susceptibility-detection model, the genetic variation may be analyzed to identify association of the genetic variation with drug resistance of the target strain with respect to a specific target drug. The susceptibility-detection model is trained based on a plurality of association mappings, wherein an association mappings associates a genetic variation in a training base strain of the pathogen with drug resistance to one or more drugs.

### BACKGROUND

A disease, specifically, an infectious disease may be caused by a pathogen, such as bacterium, virus, or any other micro-organism. Typically, an infectious disease is communicable and may be transmitted to a healthy person. In certain cases, a person may, for example, consume immune suppressants for a transplanted organ, have certain type of disease or disorder, such as HIV, AIDS, diabetes, and cancer, or have certain other medical condition, such as implanted medical devices, malnutrition and extremes of age. In such a case, an immune system of the person may not work properly thereby predisposing the person to an infectious disease, delaying recovery of the person and causing greater suffering. Another severe disease tuberculosis (also referred to as TB) is a contagious infectious disease inflicting lungs of a human being and is caused by Mycobacterium tuberculosis. TB is fatal and has reportedly claimed millions of lives globally and is considered to be one of the leading causes of death, worldwide. Patients with compromised immune systems or other underlying medical conditions, such as people living with HIV, malnutrition, or diabetes, are exposed to a higher risk of contracting TB.A Patient suffering from an infectious disease may be administered an anti-pathogen drug, which have been observed to have a substantial impact on reducing mortality associated with the disease.

Despite the presence of anti-pathogen drugs, one of the major factor that contributes to the high morbidity and mortality due to infectious diseases, is the emergence of drug resistance in pathogens. For example, owing to the slow growth rate in culture medium, identifying drug resistance in M. tuberculosis take about 4-6 weeks before a suitable drug or a combination of drugs is ascertained. As a result, choosing the best combination of drugs for a patient at an early stage of treatment becomes a challenge.

### BRIEF DESCRIPTION OF FIGURES

Systems and/or methods, in accordance with examples of the present subject matter are now described, by way of example, and with reference to the accompanying figures, in which:

FIG. 1 illustrates a system for training a susceptibility detection model, as per an example;

FIG. 2 illustrates a drug-resistance assessment system for determining drug resistance of a target strain of a pathogen, as per another example;

FIG. 3 illustrates a graphical illustration depicting the accuracy of approaches for determining drug resistance of a target strain, as per an example; and

FIG. 4 illustrates a method for training a susceptibility detection model and for determining drug resistance of a target strain of a pathogen, based on a susceptibility detection model, as per an example.

### DETAILED DESCRIPTION

As described above, ascertaining drug resistance in a given strain of a pathogen is time and a resource consuming exercise. For example, in the case of may M. tuberculosis it may take about 4-6 weeks. Subsequently, a suitable drug or a combination of drugs for the given strain of the pathogen may be ascertained only after testing the drug resistance. Drug resistance generally occurs when a strain of a pathogen does not respond to a drug. A delay in ascertaining drug resistance of the pathogen may result in ineffective drug recommendations, delayed recovery, and poor disease outcome. In certain cases, it may also result in a further spread of drug resistant pathogen.

Generally, drug resistance of a strain of a pathogen may be caused due to genetic variations or mutations within a nucleotide sequence of the strain. Some examples of such genetic variations may include single nucleotide polymorphisms (or SNPs), deletions, insertions or mutations within the nucleotide sequence of the strain under consideration. Certain approaches exists which involve correlating the genetic variations to drug-resistances. However, as may be understood, drug resistance may not occur solely owing to such genetic variations. In certain instances, such genetic variations may be compensated by some other genetic factor, owing to which the strain under consideration may still be susceptible, despite the presence of the genetic variations. Furthermore, on detecting a new genetic variation in a strain of the pathogen, conventional approaches may not provide any drug recommendation.

Approaches for ascertaining drug resistance of a target strain of a pathogen are described. The present approaches employ gene sequencing and machine learning for ascertaining drug resistance of the target strain of the pathogen. The approaches as discussed provide a rapid and comprehensive mechanism for ascertaining whether the target strain of is resistant to a drug. The determination is quick and accurate which in turn enables prescribing a correct drug in timely manner to a patient undergoing treatment pursuant to the target strain of the pathogen. Examples of the pathogen include, but are not limited to, viral, bacterial or fungal pathogens.

The present approaches are further described in the context of a training phase and a testing phase. In the context of machine learning, training involves subjecting a machine learning model with training data. Once trained, certain patterns or determinations may be made based on the training data. Such determinations are implemented within the testing phase. The machine learning model may be implemented through machine-executable code on a processor-based computing system. In an example, the machine learning model may be a susceptibility detection model based on which drug resistance of a target strain of a pathogen may be ascertained.

To this end, initially a plurality of association mappings are obtained. An association mapping may be a data set which associates a genetic marker present within a nucleotide sequence of a training base strain of a pathogen to drug resistance of the training base strain with respect to a reference drug. In this manner, different training base strains may be associated with different drug resistances with respect to different reference drugs. Such plurality of association mappings may then be utilized for training the susceptibility detection model.

In an example, the training of the susceptibility detection model may include determining a nucleotide sequence of a training base strain of the pathogen. A training base strain may have resistance against a reference drug, wherein drug resistance of the training base strain to the reference drug may be known. Thereafter, a nucleotide sequence of a reference strain of the pathogen may be determined. As may be understood, the reference strain of the pathogen may be a naturally-occurring strain that is devoid of any genetic variations or mutations. Once the nucleotide sequence of the training base strain and the reference strain is determined, the nucleotide sequence of the training base strain may be compared to the nucleotide sequence of the reference strain.

Based on the comparison, a genetic variation between the nucleotide sequence of the training base strain and the nucleotide sequence of the reference strain, may be determined. As may be understood, the variation may correspond to a mutation within the nucleotide sequence of the training base strain. In this manner, presence of any genetic variations between the nucleotide sequence of the training base strain and the reference strain of the pathogen may be determined. Once the genetic variation of the training base strain is determined, the same may be correlated with the reference drug, to which the training base strain under consideration may be resistant. The above procedure may be repeated for other training base strains, with genetic variations for the other training base strains thus determined being subsequently correlated with corresponding reference drugs. Thereafter, the correlations may be utilized for training the susceptibility detection model.

Once the susceptibility detection model is trained, it may be employed for ascertaining drug resistance of a target strain of the pathogen. To this end, a nucleotide sequence of the target strain is obtained. In an example, the nucleotide sequence of the target strain may be obtained from a test sample. Further, the nucleotide sequence of the target strain may be analysed to determine a genetic variation within the nucleotide sequence. The genetic variation may be determined based on, for example, comparison of the nucleotide sequence of the target strain with a nucleotide sequence of a reference strain of the pathogen.

Thereafter, the determined genetic variation may be analysed based on the trained susceptibility detection model. As mentioned previously, the susceptibility detection model was trained based on the plurality of association mappings that associates genetic variations within training base strains with corresponding drug resistance. To this end, the analysis of the genetic variation of the target strain by the susceptibility detection model assists in ascertaining drug resistance of the target strain with respect to a target drug. In other words, the susceptibility detection model determines the target drug to which the target strain to susceptible or not-resistant. It may be noted that drug susceptibility and drug resistance are complimentary assessments. Determination of drug resistance to one or more drugs against the pathogen may be relied on to conclude drug susceptibility to other drugs against the pathogen. Therefore, although the present approaches have been described in the context of determining drug resistance of the pathogen to one or more drugs, the same approaches may be utilized for determining susceptibility of a strain of the pathogen, to one or more drugs. Such implementations would also be within the scope of the present subject matter.

The above examples are further described in conjunction with appended figures. It may be noted that the description and figures merely illustrate the principles of the present subject matter. It will thus be appreciated that various arrangements that embody the principles of the present subject matter, although not explicitly described or shown herein, may be devised from the description, and are included within its scope. Moreover, all statements herein reciting principles, aspects, and examples of the present subject matter, as well as specific examples thereof, are intended to encompass equivalents thereof. In the figures, the left-most digit(s) of a reference number identifies the figure in which the reference number first appears. The same numbers are used throughout the figures to reference like features and components.

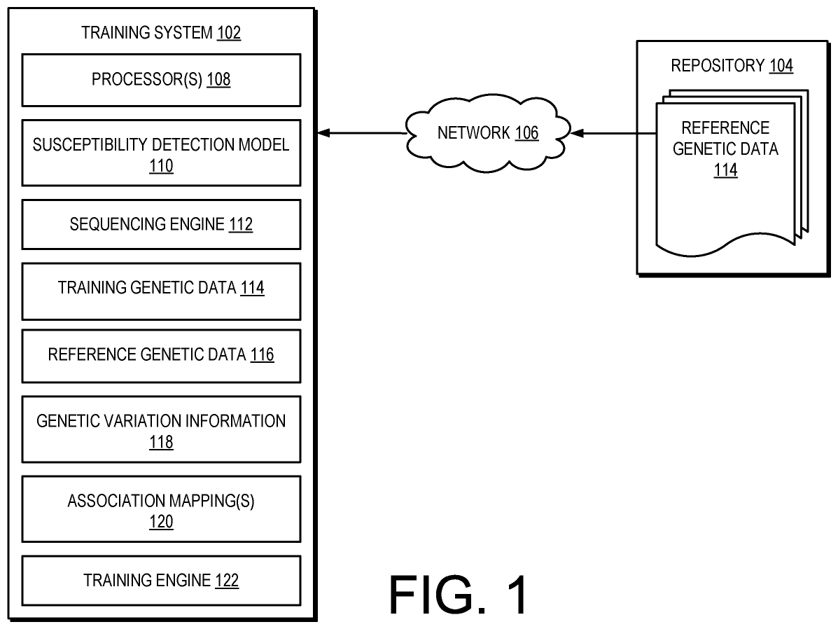

FIG. 1 illustrates a training system 102 for training a susceptibility-detection model. The susceptibility-detection model is trained based on association mappings pertaining to a plurality of training base strains of a pathogen. An association mapping of a training base strain associates a genetic marker present within a nucleotide sequence of a training base strain of the pathogen to drug resistance to a reference drug.

In an example, the training system 102 (referred to as the system 102) may be in communication with a predefined repository 104 through a network 106. The network 106 may be a private network or a public network and may be implemented as a wired network, a wireless network, or a combination of a wired and wireless network. The network 106 may also include a collection of individual networks, interconnected with each other and functioning as a single large network, such as the Internet. Examples of such individual networks include, but are not limited to, Global System for Mobile Communication (GSM) network, Universal Mobile Telecommunications System (UMTS) network, Personal Communications Service (PCS) network, Time Division Multiple Access (TDMA) network, Code Division Multiple Access (CDMA) network, Next Generation Network (NGN), Public Switched Telephone Network (PSTN), Long Term Evolution (LTE), and Integrated Services Digital Network (ISDN).

The system 102 may further include processor(s) 108 which may execute one or more computer executable instructions for training the susceptibility-detection model 110. The processor(s) 108 may be implemented as a single computing entity or may be implemented as a combination of multiple computing entities or processing units. The system 102 may further include a sequencing engine 112 and a training engine 122. The sequencing engine 112 and training engine 122 (collectively referred to as engine(s) 112, 122) may be implemented as a combination of hardware and programming, for example, programmable instructions to implement a variety of functionalities. In examples described herein, such combinations of hardware and programming may be implemented in several different ways. For example, the programming for the engine(s) 112, 122 may be executable instructions, by the processor(s) 108. Such instructions may be stored on a non-transitory machine-readable storage medium which may be coupled either directly with the system 102 or indirectly (for example, through networked means). In an example, the engine(s) 112, 122 may itself include a processing resource (not shown in FIG. 1), for example, either a single processor or a combination of multiple processors, to execute such instructions. In the present examples, a non-transitory machine-readable storage medium may store instructions, such as instructions 108, that when executed by the processing resource, implement engine(s) 112, 122. In other examples, the engine(s) 112, 122 may be implemented as electronic circuitry.

The system 102 may further include training genetic data 114, reference genetic data 114, genetic variation information 118, and association mappings 120. In operation, the system 102 may obtain training genetic data 114 from the repository 104. The repository 104 may be any implemented as any data storage repository which stores information pertaining the training genetic data 114. The training genetic data 114 may include nucleotide sequence data corresponding to a training base strain of the pathogen. The training base strain may refer to a strain of the pathogen which is known to be resistant to a reference drug. Further, the reference genetic data 114 may include nucleotide sequence data pertaining to a reference strain of the pathogen. The reference strain may refer to a naturally-occurring strain of the pathogen that is devoid of any genetic variation or mutation

In operation, the sequencing engine 112 may compare the training genetic data 114 and the reference genetic data 114 to determine occurrence of a genetic variation in the training base strain of the pathogen. To this end, the sequencing engine 112 may parse the nucleotide sequence of the training base strain to determine start and stop sequences. In certain instances, when the start and/or stop sequences relatively shift, the sequencing engine 112 may check and fix any alignment issues that may have occurred between the references strain and the reference strain. Proceeding further, the sequencing engine 112 may further compare the nucleotide sequence of the training base strain and the nucleotide sequence of the reference strain to determine a genetic variations in the training base strain. A genetic variation in the nucleotide sequence of the training base strain may manifest as a different combination of amino acids, for a given sequence location when compared with the same corresponding location in the nucleotide sequence of the reference strain.

In an example, the sequencing engine 112 may perform additional assessments, for example checking quality of the training genetic data 114, determining whether the training genetic data 114 corresponds to the entire nucleotide sequence of the training base strain, or whether the training base strain that is under consideration, is in fact that of the pathogen under consideration. Once the additional assessments are completed, the sequencing engine 112 may proceed and compare the training genetic data 114 and the reference genetic data 114 to determine and locate the genetic variations. While comparing, the sequencing engine 112 may determine the locations within the nucleotide sequence of the references strain in which combination of the amino acids differs from the combination of amino acids at a corresponding location of the nucleotide sequence of the reference strain. Once a deviation is detected, the same is flagged as a genetic variation and the corresponding information is recorded. The genetic variation may be a mutation that may be present within the training base strain.

In an example, information pertaining to the different genetic variations may be stored as genetic variation information 118 within the system 102. In addition to the deviation between the training base strain and the reference strain, the sequencing engine 112 may also determine and record certain other information. For example, the sequencing engine 112 may record a gene in which the genetic variation may have occurred. In another example, the sequencing engine 112 may also record the start sequence and the stop sequence, and a position of the variation.

Once the genetic variation is determined, the genetic variation may be correlated with a reference drug to provide association mapping(s) 120. The reference drug may be a drug to which the training base strain of the pathogen has exhibit drug resistance. The sequencing engine 112 may record a correlation between the genetic variation in the training base strain with the reference drug as genetic variation information 118. The genetic variation information may include correlations between a plurality of genetic variations in a plurality of corresponding training base strains with a plurality of corresponding reference drugs.

In an example, the pathogen may be M. Tuberculosis. In such a case, the plurality of the reference drugs for the M. Tuberculosis pathogen may be anti-TB drugs. Examples of anti-TB drugs include, but are not limited to, Isoniazid, Rifampicin, Ethambutol, Pyrazinamide, Streptomycin, Ciprofloxacin, Moxifloxacin, Ofloxacin, Amikacin, Capreomycin, Kanamycin, Prothionamide, Ethionamide, Paraaminosalicylic acid, Cycloserine, Rifabutin, Bedaquiline, Delamanid, Pretomanid and Levofloxacin. Further, an example of a genetic variation information 118 for a plurality of training base strains of the M. Tuberculosis pathogen is provided in Table A below:

TABLE A

| | | | | Mutation | |
|---|---|---|---|---|---|
| Drug | Gene | Start | Stop | Position | Mutation |
| Ciprofloxacin | gyrA | 7302 | 9818 | A74S | GCT/AGT, GCG/AGT, |
| | | | | | GCG/AGC, GCT/TCT, |
| | | | | | GCC/TCT, GCA/TCT, |
| | | | | | GCG/TCT, GCT/TCC, |
| | | | | | GCC/TCC, GCA/TCC, |
| | | | | | GCG/TCC, GCT/TCA, |
| | | | | | GCC/TCA, GCA/TCA, |
| | | | | | GCG/TCA, GCT/TCG, |
| | | | | | GCC/TCG, GCA/TCG, |
| | | | | | GCG/TCG |
| Ciprofloxacin | gyrA | 7302 | 9818 | D94G | GAT/GGT, GAC/GGA, |
| | | | | | GAC/GGG |
| Ciprofloxacin | gyrA | 7302 | 9818 | S91P | AGT/CCT, AGC/CCA, |
| | | | | | AGC/CCG |
| Ethambutol | embB | 4246514 | 4249811 | D354A | GAT/GCT, GAC/GCA, |
| | | | | | GAC/GCG |
| Ethambutol | embB | 4246514 | 4249811 | G406A | GGT/GCT, GGC/GCA, |
| | | | | | GGC/GCG, GGA/GCT, |
| | | | | | GGA/GCC, GGA/GCA, |
| | | | | | GGA/GCG, GGG/GCT |
| | | | | | GGG/GCC, GGG/GCA, |
| | | | | | GGG/GCG |
| Ethambutol | embB | 4246514 | 4249811 | G406D | GGT/GAT, GGG/GAT, |
| | | | | | GGG/GAC |
| Ethambutol | embB | 4246514 | 4249811 | G406S | GGT/AGT, GGG/AGT, |
| | | | | | GGG/AGC |
| Ethambutol | embB | 4246514 | 4249811 | M306I | ATG/ATT |
| Ethambutol | embB | 4246514 | 4249811 | M306V | ATG/GTT |
| Ethambutol | embB | 4246514 | 4249811 | Q497K | CAA/AAA |
| Ethambutol | embB | 4246514 | 4249811 | Q497R | CAA/AGA, CAG/AGA, |
| | | | | | CAG/AGG, CAG/CGT, |
| | | | | | CAG/CGA, CAG/CGC, |
| | | | | | CAG/CGG |
| Ethambutol | embB | 4246514 | 4249811 | C12T | TGT/ACT, TGT/ACG, |
| | | | | | TGC/ACG |
| Ethambutol | embB | 4246514 | 4249811 | C16G | TGT/GGT, TGT/GGG, |
| | | | | | TGC/GGG |
| Ethambutol | embB | 4246514 | 4249811 | C16T | TGT/ACT, TGT/ACG, |
| | | | | | TGC/ACG |
| Ethambutol | embB | 4246514 | 4249811 | D328Y | GAT/TAT |
| Ethambutol | embB | 4246514 | 4249811 | H1002R | CAT/CGT, CAT/CGG, |
| | | | | | CAC/CGG, CAT/AGA, |
| | | | | | CAC/AGA, CAT/AGG, |
| | | | | | CAC/AGG |
| Ethambutol | embB | 4246514 | 4249811 | N1033K | AAT/AAA |
| Isoniazid | inhA | 1674202 | 1675012 | I194T | ATT/ACT, ATC/ACA, |
| | | | | | ATC/ACG, ATA/ACT, |
| | | | | | ATA/ACC, ATA/ACA, |
| | | | | | ATA/ACG |
| Isoniazid | inhA | 1674202 | 1675012 | I21T | ATT/ACT, ATC/ACA, |
| | | | | | ATC/ACG, ATA/ACT, |
| | | | | | ATA/ACC, ATA/ACA, |
| | | | | | ATA/ACG, |
| Isoniazid | katG | 2153889 | 2156112 | S315N | AGT/AAT |
| Isoniazid | katG | 2153889 | 2156112 | S315T | AGT/ACT, AGC/ACA, |
| | | | | | AGC/ACG |
| Isoniazid | inhA | 1674202 | 1675012 | S94A | AGT/GCT, AGC/GCA, |
| | | | | | AGC/GCG |
| Isoniazid | katG | 2153889 | 2156112 | T180K | ACT/AAA, ACG/AAA, |
| | | | | | ACG/AAG |
| Isoniazid | katG | 2153889 | 2156112 | W191R | TGG/AGA |
| Isoniazid | katG | 2153889 | 2156112 | W328L | TGG/TTA |

The first column indicates a reference drug to which a training base strain, say training base strain A, is resistant. As is evident from the first row of the Table A, the training base strain A is resistant to the drug Ciprofloxacin. Table A further depicts a gene, along with a start and a stop sequence of a nucleotide sequence of the training base strain A. Furthermore, as per the first row of the Table A, the training base strain A may be resistant to the drug Ciprofloxacin due to a genetic variation present at position A74S, wherein mutations at the position A74Sis depicted in the last column. In a similar manner, other genetic variations for other references strains or same training base strains (identified by the gene column in Table A) may be recorded to provide the association mapping(s) 120. It may be noted that the Table A is just one of the many possible examples. The association mapping(s) 120 may include additional genetic variation information 118 or may include further entries which correlate the genetic variation information 118 with other types of drugs. Such other examples would also be within the scope of the present subject matter. Although the present example has been explained with respect to the M. tuberculosis, the present approaches may be implemented without limitation for any other pathogen, such as a virus, other bacteria or fungi.

Returning to the present example, the training engine 122 may then train the susceptibility detection model 110 based on the association mapping(s) 120. In an example, the susceptibility detection model 110 may be based on a number of classification or regression-based learning techniques. An example of such a technique includes random forest. Other examples may also be utilized for implementing the susceptibility detection model 110, without deviating from the scope of the present subject matter. In an example, the susceptibility detection model 110 may be in the form of a classifier.

Once the susceptibility detection model 110 is trained, it may be utilized for determining whether a target strain of the pathogen is resistant (or susceptible) to a target drug. To this end, the trained susceptibility detection model 110 may be implemented within a computing system for assessing drug resistance. The system may analyze the target strain to determine whether the target strain is resistant to the target drug.

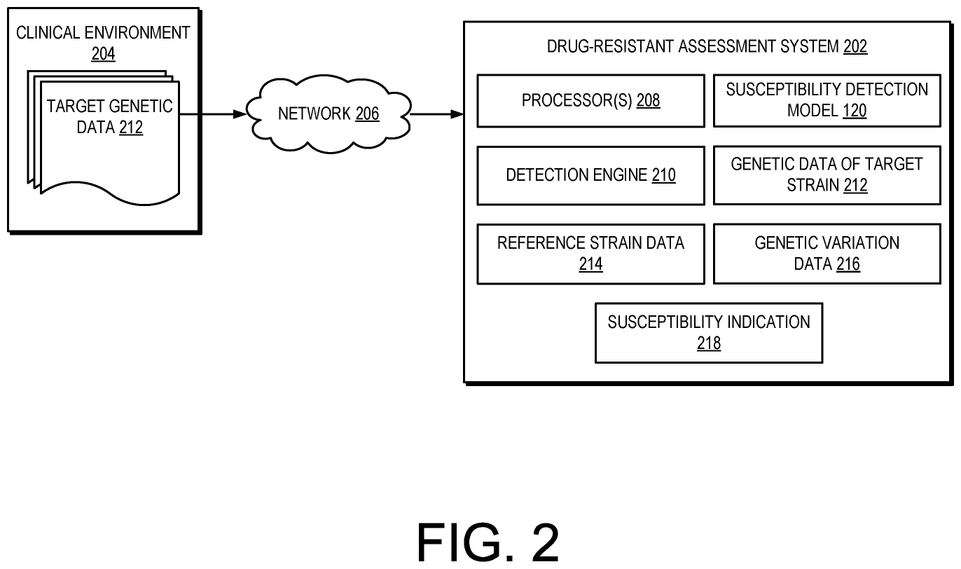

An example of such a computing system is described in conjunction with FIG. 2. FIG. 2 depicts a drug-resistance assessment system 202 (hereinafter referred to as the assessment system 202). The assessment system 202 may be in communication with a clinical environment 204 over a communication network 206. In an example, the communication network 206 is similar to the network 106. The clinical environment 204 may be an environment which may be testing one or more test samples acquired from one or more patients. The clinical environment 204 may include any facility or institution implementing mechanisms for retrieving and storing target genetic data 208 of a target strain in computer-accessible storage devices. Such storage devices may either be within the premises of the clinical environment 204 or may be remotely accessible by one or more computing devices within the clinical environment 204. In an example, the assessment system 202 may also be implemented within the clinical environment 204 without deviating from the scope of the present subject matter.

Returning to the present example, the assessment system 202 may further include a detection engine 210. The detection engine 210, based on the susceptibility detection model 110 is to determine the drug resistance of the target strain. In one example, genetic data of the target strain (referred to as target genetic data 212) is obtained. The target genetic data 212 may be based on patient samples that may be collected by the clinical environment 204. In an example, the patient sample may be specifically processed to extract a target strain of M. tuberculosis pathogen is to be assessed. Once the target strain is extracted, the extracted DNA may be validated for quality and quantity.

After the validation of the target genetic data, it may be processed to determine a nucleotide sequence of the target strain under consideration. The nucleotide sequence information may then be determined and transmitted to the assessment system 202. The nucleotide sequence information may then be stored in the assessment system 202 as the target genetic data 212.

The target genetic data 212 may then be processed by the detection engine 210 based on the susceptibility detection model 110. Prior to the processing based on the susceptibility detection model 110, the detection engine 210 may initially determine whether a genetic variation is present within the nucleotide sequence of the target strain. To this end, the detection engine 210 may compare the nucleotide sequence of the target strain (available as target genetic data 212) of the pathogen with a nucleotide sequence of a reference strain of the pathogen (available as reference strain data 214). To compare, the detection engine 210 may parse the nucleotide sequence of the target strain and the nucleotide sequence of the reference strain to determine their corresponding start and stop sequences. Thereafter, the detection engine 210 may compare the target genetic data 212 and the reference strain data 214 to identify a genetic variation such as, a genetic marker or a genetic mutation, that may be present in the target strain. The determined genetic variation may be stored as genetic variation data 216. It may be noted that similar to the genetic variation information 118 which indicated the genetic variations between the training base strain and the reference strain, the genetic variation data 216 indicates the genetic variations between the target strain and the reference strain.

For example, a target genetic data, a training base strain data (such as the training genetic data 114) and/or a reference genetic data (such as the reference genetic data 114) may be generated based on gene sequencing techniques. In the context of the present example, the term sequencing refers to the determination of a nucleotide sequence of an amplified nucleic acid obtained. Such amplified nucleic acid may be obtained from a mycobacterial nucleic acid sample by, for example, polymerase chain reaction (PCR), or multiplex PCR. Various methods are known in the art for carrying out gene sequencing using conventional or next generation sequencing, (e.g. Sanger dideoxy, Illumina, IonTorrent, and Nanopore). It may be noted that the present examples for gene sequencing techniques are only illustrative and should not be construed to be limiting the scope of the claimed subject matter. In an example, the target genetic data, the reference genetic data and/or the wild-type genetic data may be stored in a computer-readable format.

Using the susceptibility detection model 110, the detection engine 210 may further process the genetic variation data 216. The susceptibility detection model 110 may be previously trained based on a plurality of association mappings, wherein an association mapping correlates a genetic variation in a training base strain with a reference drugs to which the training base strain may be resistant. on receiving the genetic variation data 216, the trained susceptibility detection model 110 indicates one or more target drugs to which the target strain may be resistant (or susceptible) to.

Information pertaining to the drug resistance of the target strain may be stored as susceptibility indication 218. In an example, the susceptibility indication 218 may be then communicated to a medical practitioner for prescribing either a single drug or a combination of drugs for a treatment pursuant to the target strain. In another example, the detection engine 210 may further generate a report which provides an indication of one or more drugs to which the target strain under consideration may be resistant to or susceptible to for aid in treatment against the pathogen. The report may be stored within a database or may be shared electronically with the patient or any other medical practitioner for further consideration.

Once the susceptibility indication 218 is obtained, the same may be included as part of the training genetic data 114. In this manner, the training genetic data 114 may be updated based on any new, i.e., previously unobserved genetic variations in the strains of the pathogen. Subsequently, the susceptibility detection model 110 may be trained based on the updated training genetic data 114 which will further enable determining drug resistance of the target strain (now a training base strain) of the pathogen.

It may be noted that the present approaches enable determination of drug resistance within a few hours as opposed to 4-6 weeks, which was the time taken for determining drug resistance through the culture method. It will enable faster and increased positive treatment outcomes, thereby reducing the spread and incidence of infection. Early detection of drug resistance may result in beneficial impact onto public health in relation to infectious and communicable diseases, typically caused due to bacterium. the present approaches may also optimize limited treatment options Furthermore, the early determination of drug resistance of a strain of the pathogen through the present approaches has also been highly accurate.

In an example, the present approaches for determining drug resistance may be used for treatment of Tuberculosis (TB) caused due to M. Tuberculosis. To this end, drug resistance of a target strain may be assessed based on antibacterial drugs for TB comprising one of Isoniazid, Rifampicin, Ethambutol, Pyrazinamide, Streptomycin, Ciprofloxacin, Moxifloxacin, Ofloxacin, Amikacin, Capreomycin, Kanamycin, Prothionamide, Ethionamide, Paraaminosalicylic acid, Cycloserine, Rifabutin, Bedaquiline, Delamanid, Pretomanid and Levofloxacin.

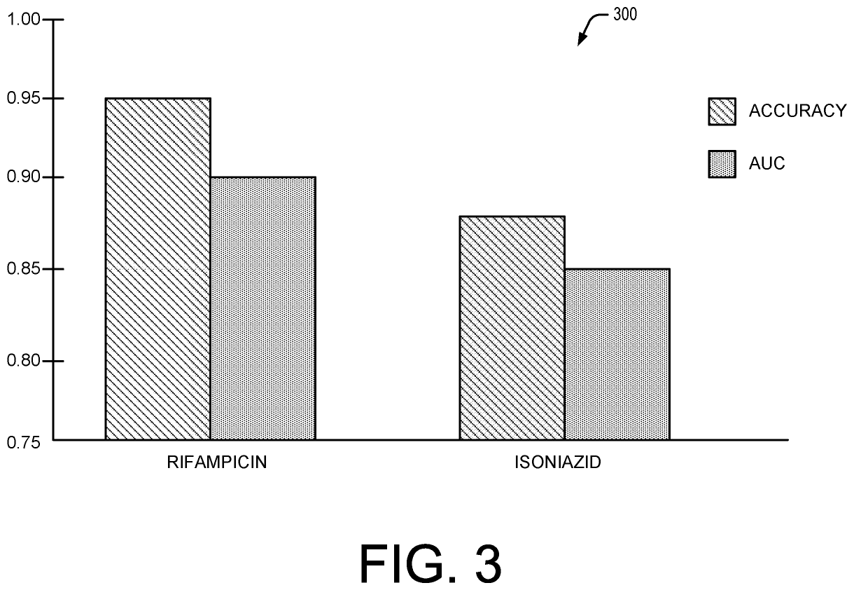

FIG. 3 provides a graphical illustration 300 depicting an accuracy of approaches for determining drug resistance of a target strain. With regard to FIG. 3, the approaches for determining drug resistance may be applied onto a target strain of an M. tuberculosis. Further, the target strain of the M. tuberculosis pathogen may be assessed to determine drug resistance against anti-TB drugs Rifampicin and Isoniazid. To this end, the techniques described above are used to obtain outcomes, i.e., drug resistance against Rifampicin and Isoniazid, for 1415 target strains. Such outcomes of the 1415 target strains are plotted as 1415 data points corresponding. As depicted, an accuracy of more than 90% has been achieved with regard to assessment of the target strains for drug resistance against Rifampicin and an accuracy of more than 85% has been achieved with regard to assessment of the target strains for drug resistance against Isoniazid. It may be noted that the graphical depiction 300 is only depicting one example and should not be construed to be a limitation on the claimed subject matter.

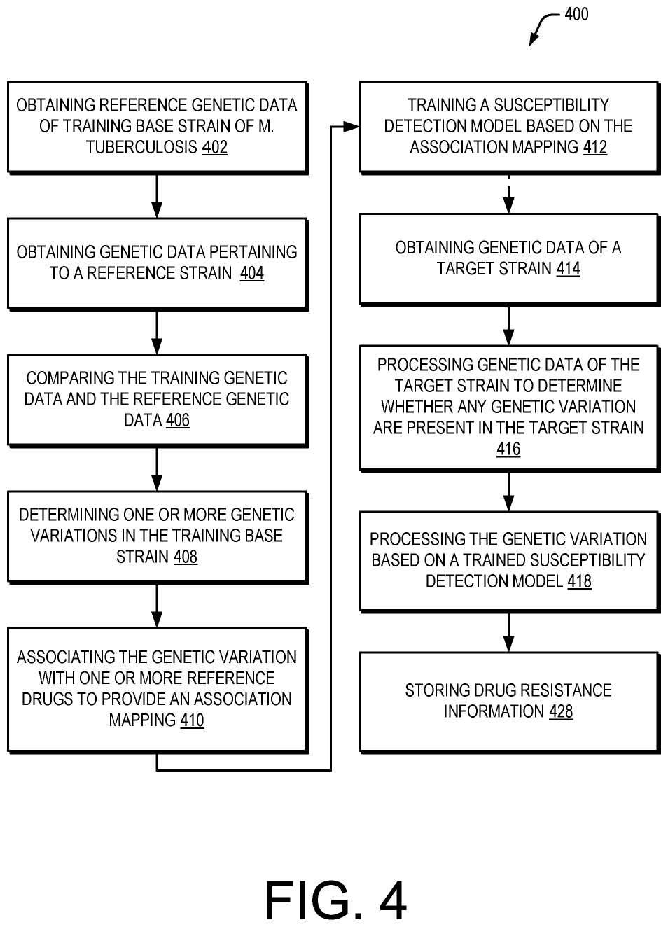

FIG. 4 illustrates a method 400 to be implemented for determining drug resistance of a target strain against a target drug, as per an example of the present subject matter. Although the method 400 may be implemented in a variety of computing devices, for the ease of explanation, the present description of the example method 400 is provided in reference to the above-described training system 102 and the assessment system 202. The order in which the various method blocks of method 400 are described, is not intended to be construed as a limitation, and any number of the described method blocks may be combined in any order to implement the method 400, or an alternative method. It may also be noted that method 400 pertains to initially training a susceptibility detection model, such as the model 110, and then subsequently determining whether a target strain is resistant to a given target drug. However, such steps may be performed separately at different instances without limiting the scope of the present subject matter in any manner.

Furthermore, the above-mentioned methods may be implemented in a suitable hardware, computer-readable instructions, or combination thereof. The steps of such methods may be performed by either a system under the instruction of machine executable instructions stored on a non-transitory computer readable medium or by dedicated hardware circuits, microcontrollers, or logic circuits. Herein, some examples are also intended to cover non-transitory computer readable medium, for example, digital data storage media, which are computer readable and encode computer-executable instructions, where said instructions perform some or all the steps of the above-mentioned methods.

At block 402, for training a susceptibility detection model, initially a reference genetic data of a training base strain of a pathogen, may be obtained. For example, the system 102 may obtain the training genetic data 114 from the repository 104 (as shown in FIG. 1). The training genetic data 114 includes a nucleotide sequence data corresponding to the training base strain of the pathogen, which is known to be resistant to a reference drug.

At block 404, genetic data pertaining to a reference strain is obtained. For example, the system 102 may obtain reference genetic data 114 wherein the reference genetic data 114 includes nucleotide sequence data pertaining to a reference strain of the pathogen. The reference strain may be a strain of the pathogen as it occurs in nature and may be devoid of any genetic variations or mutations.

At block 406, the reference genetic data and the reference genetic data are compared. At block 408, a genetic variation may be determined based on the comparing. For example, the sequencing engine 112 may compare the training genetic data 114 and the reference genetic data 114 to determine occurrence of the genetic variation in the training base strain. For example, the genetic variation may manifest in the training genetic data 114 as a different combination of amino acids when compared with the reference genetic data 114, at a given location. In an example, the genetic variation may be stored as genetic variation information 108.

At block 410, the genetic variation is associated with one or more reference drugs. The reference drugs are such drugs to which the training base strain of the pathogen may be resistant. For example, the sequencing engine 112 may correlate the genetic variation information 108 with the corresponding reference drugs to provide association mapping(s) 120. The association mapping(s) 120 may include a correlation between the genetic variation (as indicated in the genetic variation information 108) in the training base strain and one or more reference drugs to which the training base strain may be resistant to. In an example, the association mapping(s) 120 may be in form of a table, such as Table A.

At block 412, a susceptibility detection model is trained based on the association mappings. For example, the training engine 122 may train the susceptibility detection model 110 based on the association mapping(s) 120. The susceptibility detection model 110 may be based on a number of classification or regression-based learning techniques, such as random forest techniques. In an example, the susceptibility detection model 110 may be in the form of a classifier. Once the susceptibility detection model 110 is trained, it may be utilized for determining drug resistance (or susceptibility) of a target strain of the pathogen with regard to a target drug. The manner in which drug resistance of the target strain may be assessed is described in the following method blocks. As described previously, the steps of determining drug resistance of the target strain may not immediately follow the steps describing the training of the susceptibility detection model 110.

Proceeding further, at block 414, genetic data of the target strain may be obtained. For example, an assessment system 202 may obtain a target genetic data 212 from a clinical environment 204. The target genetic data 212 may be based on a target strain of the pathogen obtained from a test sample collected from a patient. In an example, the obtained target genetic data 212 may include a nucleotide sequence of the target strain. The nucleotide sequence and other information pertaining to the target strain may be stored as target genetic data 212.

At block 416, the target genetic data of the target strain may be processed to determine a genetic variation in the target strain. In an example, a detection engine 210 may determine a genetic variation within the nucleotide sequence of the target strain. In such a case, the detection engine 210 may compare the nucleotide sequence of the target strain (available as target genetic data 212) with a nucleotide sequence of a reference strain of the pathogen (available as reference strain data 214). Based on the comparing, the detection engine 210 may identify the genetic variation that may be present in the target strain. The genetic variation once determined may be stored as genetic variation data 216.

At block 418, the genetic variation may be processed based on a trained susceptibility detection model. For example, the detection engine 210 may process the genetic variation data 216 based on the susceptibility detection model 110. The trained susceptibility detection model 110, based on the genetic variation data 216, may indicate one or more target drugs to which the target strain may be resistant.

At block 420, the drug resistance information may be stored for further consideration. For example, the information pertaining to the drug resistance may be stored as susceptibility indication 218. The susceptibility indication 218 may be then communicated to a medical practitioner for prescribing either a single drug or a combination of drugs a treatment against the pathogen in the patient from which the test sample (or the target strain) was collected. In another example, the detection engine 210 may further generate a report which provides an indication of drugs to which the target strain under consideration may be resistant to or susceptible to for aid in treatment against the pathogen. The report may be stored within a database or may be shared electronically with the patient or any other medical practitioner for further consideration.

Although examples for the present disclosure have been described in language specific to structural features and/or methods, it is to be understood that the appended claims are not necessarily limited to the specific features or methods described. Rather, the specific features and methods are disclosed and explained as examples of the present disclosure.

I/We claim:

1. A method for training a processor-based system for ascertaining drug resistance of a target strain of a pathogen, the method comprising:
obtaining a plurality of association mappings, wherein an association mapping, selected from the plurality of association mappings, associates a genetic marker of a training base strain of the pathogen to drug resistance with respect to a reference drug; and
based on the plurality of association mappings, training the processor-based system to determine association of drug resistance of the target strain of the pathogen with respect to a target drug based on presence of a target genetic marker.

2. The method as claimed in in claim 1, wherein training the processor-based system comprises:
obtaining a nucleotide sequence of the training base strain of the pathogen;
comparing the nucleotide sequence of the training base strain with a nucleotide sequence of a reference strain of the pathogen;
based on the comparing, determining a variation between the nucleotide sequence of the training base strain and the nucleotide sequence of the reference strain;
correlating an indication of the variation between the nucleotide sequence of the training base strain and the nucleotide sequence of the reference strain with the reference drug; and
training the processor-based system based on the indication.

3. The method as claimed in claim 2, wherein the variation between the nucleotide sequence of the training base strain and the nucleotide sequence of the reference strain is due to a mutation in the training base strain.

4. The method as claimed in claim 2, wherein the indication is prescribed in text-based format used for representing one of nucleotide sequences and amino acid sequences.

5. The method as claimed in claim 1, wherein the training is further based on a set of clinical parameters.

6. The method as claimed in claim 1, further comprising validating the trained processor-based system based on a predefined repository of association mappings which correlate each of a plurality of genetic markers of a corresponding plurality of training base strains with one of corresponding drug resistance and corresponding drug susceptibility, with respect to a set of reference drugs.

7. The system as claimed in claim 1, wherein the target genetic marker is indicative of a mutation on the target strain.

8. A system for ascertaining drug resistance of a target strain of a pathogen, the system comprising:
a detection engine, wherein the detection engine is to:
obtain a nucleotide sequence data of the target strain of the pathogen, wherein the target strain is obtained from a test sample;
analyze the nucleotide sequence data to locate a genetic variation in a nucleotide sequence of the target strain; and
analyze the genetic variation to identify association of the genetic variation of the target strain with drug resistance with respect to a target drug based on a susceptibility-detection model, wherein the susceptibility-detection model is trained based on a plurality of association mappings, with each of the plurality of association mappings associating a genetic variation of a training base strain with a drug resistance to one or more drugs.

9. The system as claimed in claim 8, wherein the detection engine, on determining drug susceptibility of the target strain with respect to the target drug, is to cause generation of a report indicating a prospective treatment based on the target drug.

10. The system as claimed in claim 8, wherein the detection engine is to locate the genetic variation based on comparison of the nucleotide sequence data of the target strain with nucleotide sequence data of a reference strain.

11. The system as claimed in claim 8, wherein the nucleotide sequence data of the target strain is obtained from a test sample.

12. The system as claimed in claim 8, wherein the pathogen is Mycobacterium tuberculosis.

13. The system as claimed in claim 12, wherein the target drug is an antibacterial drug comprising one of Isoniazid, Rifampicin, Ethambutol, Pyrazinamide, Streptomycin, Ciprofloxacin, Moxifloxacin, Ofloxacin, Amikacin, Capreomycin, Kanamycin, Prothionamide, Ethionamide, Paraaminosalicylic acid, Cycloserine, Rifabutin, Bedaquiline, Delamanid, Pretomanid and Levofloxacin.

14. A non-transitory computer-readable medium comprising computer-readable instructions, which when executed by a processor of a computing device, cause the processor to:
obtain a nucleotide sequence data of a target strain of a pathogen obtained from a test sample;
analyze the nucleotide sequence data to locate a mutation in a nucleotide sequence of the target strain; and
determine drug resistance of the target strain with respect to a target drug by analyzing the mutation based on a susceptibility-detection model, wherein the susceptibility-detection model is trained based on a plurality of association mappings, with each of the plurality of association mappings associating a mutation of a training base strain with drug resistance to one or more drugs.

15. The non-transitory computer-readable medium as claimed in claim 13, wherein the instructions are to further:
determine whether the located mutation is recorded in a predefined repository;
in response to determining that the located mutation is unrecorded in the predefined repository, flagging the mutation of the target strain; and
cause training of the susceptibility-detection model based on the flagged mutation, on identifying the target strain as one of a training base strain.
